(19) United States
(12) Patent Application Publication
(10) Pub. No.: US 2008/0287829 A1
(43) Pub. Date: Nov. 20, 2008

(54) COLLECTION MEANS AND A METHOD FOR COLLECTING CORD BLOOD

(76) Inventors: Thomas E. Moore, Tucson, AZ (US); Beth Mapother, Tucson, AZ (US)

Correspondence Address: PILLSBURY WINTHROP SHAW PITTMAN LLP, P.O. BOX 10500, MCLEAN, VA, 22102, US

(21) Appl. No.: 12/062,500

(22) Filed: Apr. 3, 2008

Related U.S. Application Data
(63) Continuation of application No. 11/186,415, filed on Jul. 20, 2005, now abandoned.
(60) Provisional application No. 60/590,386, filed on Jul. 20, 2004.

(51) Int. Cl. A61B 5/153

(52) U.S. Cl. 600/573; 206/569

(57) ABSTRACT

The invention provided herein comprises a kit, where the kit includes at least one collecting receptacle for collecting cord blood, and a pre-measured non-liquid anticoagulant which is optionally pre-loaded into the receptacle. The anticoagulant is preferably selected from the group consisting of heparin or heparin type additives, citrates, EDTA (ethylenediaminetetraacetic acid), and oxalates and any other anticoagulant that can be used to decrease the clotting ability of the blood. The invention provided herein also comprises a method for collecting cord blood, where the method comprises collecting a desired amount of cord blood from the umbilical cord using a collection receptacle of the invention, wherein the collection receptacle is pre-loaded with a pre-measured amount of non-liquid anticoagulant.

This application is a continuation of U.S. patent application Ser. No. 11/186,415, filed Jul. 20, 2005 entitled “Collection Means and a Method for Collecting Cord Blood”, which claims priority to U.S. Provisional Application No. 60/590,386 entitled “Collection Means and a Method For Collecting Cord Blood” filed on Jul. 20, 2004, which applications are expressly incorporated by reference herein.

### BACKGROUND OF THE INVENTION

1. Field of the Invention

The invention relates to a kit and method directed towards the collection of cord blood.

2. Background of the Invention

Cord blood, which is also known as “placental blood,” is the blood that remains in the umbilical cord and placenta after birth and after the cord is cut. Typically, cord blood is discarded with the placenta and the umbilical cord. Fetal blood is a source of numerous blood factors that have important commercial and therapeutic uses in many fields, including tissue culture, bone marrow transplantation, stem cell collection, pharmacology, and biologic research. For example, it has been determined, however, that a baby's umbilical cord blood is a valuable source of stem cells and other mononucleated cells.

Stem cells are the primitive cells from which other blood cells—white cells, red cells and platelets—develop; hence they are the building blocks of the immune and blood cell systems. There are three sources which provide stem cells: bone marrow, peripheral blood, and umbilical cord blood. Cord blood stem cells are unique in that they are immature “naive” immune cells. The differentiating ability of cord blood stem cells provides significant promise for improving treatment of some of the most common diseases, such as heart disease, stroke, and Alzheimer's disease. Moreover, when they are used in transplantation, they have reduced reactivity and are less likely to consider the recipient foreign. Further, when transplanted, even a single stem cell has the ability to regenerate the person's blood system. Hence, they are vital for weakened immune systems.

Because there are so many uses for cord blood, and because the number of cells transplanted correlates with time to engraftment, the maximum possible amount of the blood should be collected and stored. Unfortunately, however, the present methods are not optimal for collecting cord blood.

Previously, cord blood had been collected using various collection receptacles with liquid heparin or liquid sodium citrate as an anticoagulant. The anticoagulant was added to the collection receptacle by the physician or nurse prior to the cord blood collection in the operating/delivery room. Alternatively, non-sterile bags with pre-loaded citrate-phosphate-dextrose anticoagulant (CPD) are also commonly used. These bags require a sterile extension to be used at the time of collection. As the receptacle must be sterile at the time of use, and as the sterilization process damages the liquid anticoagulant, pre-filling the receptacle with the liquid anti-coagulant was not desirable. On the other hand, manually adding the anticoagulant required an additional step by the cord blood collector (i.e. the physician or nurse) and thus introduced an additional risk, for example, potential failure to add the desired amount or any anticoagulant (which would lead to clotting of the collected blood) or adding too much anticoagulant, thereby reducing the value of the cord blood supply. In addition, the introduction of liquid anticoagulant resulted in lower yields (defined by the number of mononuclear cells available after processing divided by the number of such cells collected).

Another problem with the collection of cord blood is that because the environment of a delivery room is biologically diverse and with a variety of contaminants such as maternal blood and urine present on the umbilical cord and in the surrounding area, it is desirable to have an apparatus and method for collecting cord blood in an aseptic manner.

Accordingly, it is desirable to have a convenient apparatus and method for collecting cord blood in an aseptic or sterile environment without the risk observed when manually adding anticoagulants immediately prior to use in environments that are not both sterile and intended for performance repeatability or when using liquid anticoagulants.

### BRIEF DESCRIPTION OF THE DRAWINGS

Aspects and features of the present invention will become apparent to those ordinarily skilled in the art upon review of the following description of specific embodiments of the invention in conjunction with the accompanying figures, wherein:

FIG. 1 illustrates a kit that includes a syringe according to the present invention; and

FIG. 2 illustrates a kit that includes a bag according to the present invention.

### DETAILED DESCRIPTION OF THE PREFERRED EMBODIMENTS

The invention provided herein comprises a kit, where the kit includes at least one collecting receptacle for collecting cord blood, and a pre-measured non-liquid anticoagulant which is optionally pre-loaded into the receptacle. The invention provided herein also comprises a method for collecting cord blood, where the method comprises collecting a desired amount of cord blood from the umbilical cord using a collection receptacle of the invention, wherein the collection receptacle is pre-loaded with a pre-measured amount of non-liquid anticoagulant. “Cord” and “umbilical cord” are used interchangeably herein. Similarly, “cord blood” and “umbilical cord blood” are used interchangeably herein.

The collection receptacle can be in the form of a syringe, bag, beaker or pouch or any other container, including non-flaccid (e.g., semi-rigid) containers, or means for collecting cord blood and of sufficient size to collect the desired amount of cord blood. Sufficient size means large enough but not substantially larger than is necessary to receive the desired amount of cord blood. When using more than one collection receptacle, any combination of collection receptacles may be used.

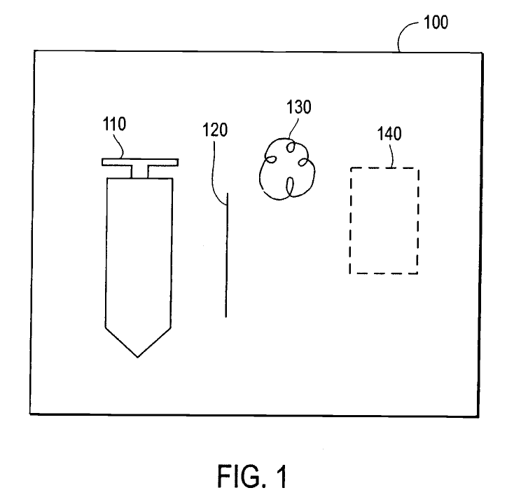

FIG. 1 illustrates a kit 100 that includes a syringe 110, a needle 120 for use with syringe, and a sterile wipe 130. Other components can also be inserted, shown as a box 140, and there other components can be vacutainers for maternal blood collection, trays, identification labels, instructions, gauze pads, needle holders, etc.

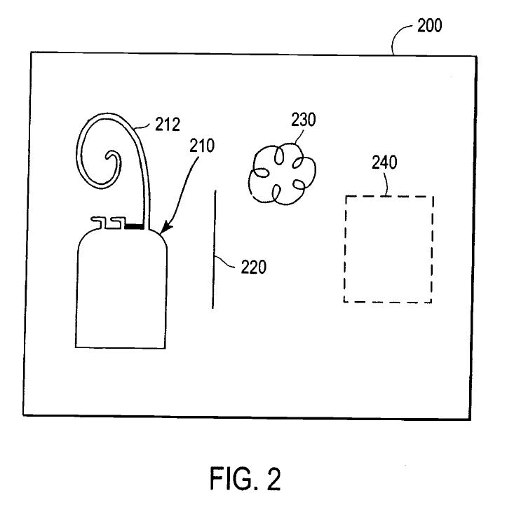

FIG. 2 illustrates a kit 200 that includes a collection bag 210, which collection bag 210 preferably has attached to it at the manufacturing facility a tube 212 through which the cord blood can flow when being collected. Also included in the kit is a needle 220 and other components shown as box 230 which other components can be vacutainers for maternal blood collection, trays, identification labels, instructions, gauze pads, needle holders, etc.

The anticoagulant is preferably selected from the group consisting of heparin or heparin type additives, citrates, EDTA (ethylenediaminetetraacetic acid), and oxalates and any other anticoagulant that can be used to decrease the clotting ability of the blood. For example, other anticoagulants that may be used in the invention include warfarin, acenocoumarol, dicumarol, anisindione, and lepirudin. More preferably heparin or sodium citrate is used.

The anticoagulant is in the form of a non-liquid, preferably lyophilized or in powder form.

The invention has several advantages, including repeatability of the components of the kit and sterility of the components therein, convenience of collection, elimination of errors in determining the amount of anticoagulant to use and in the case of preloaded anticoagulant, eliminating the possibility of the collector's failure to add the anticoagulant. Using the lyophilized or other non-liquid coagulant also allows for increased yields on processing the cord blood sample, as can be seen in the attached data. This is a result of the osmolality of the anticoagulated cord blood sample when liquid is added as opposed to the osmolality of the anticoagulated cord blood sample when the blood itself acts as the diluent for the non-liquid anticoagulant. Moreover, the use of a non-liquid anticoagulant provides for uniform distribution of the anticoagulant through the cord blood sample. Yet another advantage of using non-liquid anticoagulant is that the flexibility of storage temperature. Specifically, liquid anti-coagulant, such as heparin, should preferably be stored at 25° C. In contrast, the temperature parameters for storing non-liquid heparin as used in the invention can be anywhere from about 0° C. to about 30° C. Further, and as seen in Table 3, the amount of blood collected with the non-liquid anti-coagulant is greater than with liquid anti-coagulant. Accordingly, the invention provides not only for higher yields but also for greater collection volume than what was previously known.

Moreover, the kit provides for a sterilized means for collecting cord blood. The kit itself is prepared in a sterile manufacturing environment that allows for repeatability of correct procedures, particularly mounts that are preloaded into the container, as well as sterility of components in the kit. Further, inspection can occur at the manufacturing environment.

In the case of syringes, inspection can include whether there is a complete lid seal, no syringe cracks, and no missing components. The syringes are preferably pre-filled with the desired amount of non-liquid anticoagulant sufficient to prevent substantial clotting of the later collected cord blood, preferably 5 or about 5 heparin pellets per syringe (i.e., 0.03 grams or about 0.03 grams of heparin). Once the kit has been prepared with the syringe and any other desired components, the complete kit is sterilized, using known sterilization techniques. Hence, the possibility of contamination or spillage of the pre-filled non-liquid anticoagulant is minimized or eliminated.

When using kits with bags, the bags are previously sterilized and are then preferably filled with the desired amount of non-liquid anticoagulant sufficient to prevent substantial clotting of the later collected cord blood, preferably 5 or about 5 heparin pellets per bag (i.e., 0.03 grams or about 0.03 grams of heparin). The kit is similarly prepared in a sterile manufacturing environment. This allows for inspection that can include whether there are no kinks, in the tubing that is attached to the bags, tears or cuts in either the bag or attached tubing, missing parts and that there is a complete lid seal. Once the kit has been prepared with the bag and any other desired components, the complete kit is sterilized, using known sterilization techniques. Hence, the possibility of contamination or spillage of the pre-filled non-liquid anticoagulant is minimized or eliminated.

### Syringes:

To obtain the cord blood using syringes, the cord is clamped and cut close to the infant. After the cord is swabbed with a sterilizer such as Betadine® or alcohol, the blood is aspirated starting from the fetal end of the umbilical vein using the syringe 110 and attached needle 120, illustrated separately in FIG. 1, or other collection receptacle of the invention, taking advantage of any uterine contractions. When using multiple syringes, each syringe should be inserted farther up the cord. The procedure may be repeated as often as is necessary to collect a desired amount of cord blood. After collection, the syringe is preferably inverted back and forth to mix the anticoagulant.

### Collection Bags:

To obtain the cord blood using collection bags such as bag 210 illustrated in FIG. 2, the cord is clamped and cut close to the infant. After the area surrounding the insertion site on the cord is swabbed with a sterilizer such as Betadine® or alcohol, the needle 220 attached to the tubing 212 of the collection bag 210 is inserted the cord at the prepared insertion site. To maximize flow rate, the bag 210 is preferably maintained at a level lower than the insertion site for gravity assisted drainage. It is preferable that the big be moved back and forth in a rocking manner to mix the anticoagulant with the blood as it is being collected. After the blood flow has stopped, it is desirable to milk the remaining blood in the tubing to collect the maximum amount of cord blood. The “milking” process is performed by the physician placing the index finger and thumb around the umbilical cord as close as possible to the insertion of the cord into the placenta proper and gently pulling downward so as to force any remaining cord blood to extrude out of the distal end. The procedure may be repeated as often as is necessary to collect a desired amount of cord blood.

The invention has several advantages over products for collection of cord blood which use liquid heparin. First, convenience of storage of vials with liquid heparin is less than that of vials with non-liquid heparin, e.g. lyophilized heparin. For example, vials with liquid heparin should be stored at 25° C., though deviations are permitted in the range of 15° C.-30° C. In contrast, vials with non-liquid heparin can be stored at any temperature preferably between about 0° C. to about 30° C.

Moreover, in small volumes of cord blood (e.g., volumes of about 30 ccs or less), the percent recovery of mononucleated cells (MNC) is extremely important. When using non-liquid heparin, e.g., lyophilized heparin, the % MNC recovery is at least about 95% whereas with liquid heparin, the % recovery was shown to be about 85%. One possible reason for this may be because osmolality does become a factor until the liquid heparin represents at least about 25% of the total liquid per container. Osmolality is a count of the total number of osmotically active particles in a solution and is equal to the sum of the molalities of all the solutes present in that solution.

The following tables provide data showing the benefits of the use of non-liquid, including lyophilized, anticoagulants in cord blood collection. Table 1 shows the validation of sterile bags with heparin pellets. Table 2 shows yield data from dry (lyophilized) heparin. Table 3 shows collections volumes from CPD bags vs. lyophilized heparin bags for >30 cc collections. Table 4 shows pre and post processing data for lyophilized heparin collections. Table 5 (including the chart) shows pre and post processing data for liquid heparin in syringe and volume collection histogram. Table 6 (including the chart) shows pre and post processing data for lyophilized heparin in syringe and volume collection histogram. Table 7 (including the chart) shows pre and post processing data for liquid heparin in bag and volume collection histogram. Table 8 (including the chart) shows pre and post processing data for lyophilized heparin in bag and volume collection histogram. Table 9 shows pre and post processing data for CPD anticoagulant in bags. Table 10 shows a comparison of low volume collections in syringes with liquid heparin and lyophilized heparin.

Although the present invention is described with respect to certain preferred embodiments, modifications thereto will be apparent to those skilled in the art, and from the scope of the claims set forth below.

### EXAMPLES

TABLE 1
Validation of sterile bags with heparin pellets
Beginning Sep. 25, 2003 through Oct. 04, 2003

| | | | Post | |
|---|---|---|---|---|
| Sample | Volume | Pre TNC | TNC | % MNC |
| ID | (CC) | (10\hat{ }6) | (10\hat{ }6) | Recovery |
| 8 . . . 171 | 25 | 178.8 | 105.5 | 94.4 |
| 8 . . . 172 | 26 | 520 | 459.5 | 99.3 |
| 8 . . . 173 | 50 | 446 | 369.5 | 87.9 |
| 8 . . . 174 | 120 | 968.4 | 381.5 | 70.5 |
| 8 . . . 175 | 15 | 123.6 | 79.5 | 105.4 |
| 8 . . . 185 | 54 | 1074.6 | 425 | 89.6 |
| 8 . . . 186 | 62 | 626.2 | 370 | 89.91 |
| 8 . . . 187 | 46 | 662.4 | 389 | 94.6 |
| 8 . . . 188 | 120 | 1512 | 735 | 95.05 |
| 8 . . . 189 | 16 | 190.4 | 103.5 | 95.1 |
| 8 . . . 190 | 17 | 165.8 | 89.5 | 82.51 |
| 8 . . . 191 | 4 | 29.8 | 11.5 | 50.35 |
| 8 . . . 192 | 41 | 487.9 | 244.5 | 91.13 |
| 8 . . . 193 | 102 | 1193.4 | 424 | 86.5 |
| 8 . . . 194 | 20 | 142.4 | 44.3 | 73.71 |
| 8 . . . 195 | 54 | 626.4 | 314.5 | 91.62 |
| 8 . . . 198 | 71 | 837.8 | 437 | 86.25 |
| 8 . . . 199 | 12 | 127.2 | 131 | 124.66 |
| Average | 47.5 | 550.7278 | 284.1278 | 89.36056 |

TABLE 2
Yield Data from Dry (Lyophilized) Heparin

| | | Pre-Count | | | | | Post-Count | | | | | | | |
|---|---|---|---|---|---|---|---|---|---|---|---|---|---|---|
| | | Total | | | | MNC | Total | | | | MNC | % | | |
| Sample* | Volume | NC | Cells/mL | Diff. | MNC | Total | NC | Cells/mL | Diff. | MNC | Total | Rec | Notes | |
| 47 | 43 | 447 | 10.4 | 0.508 | 227 | 227 | 255 | 5.9 | 0.875 | 223 | 223 | 98 | | 1 |
| 48 | 57 | 644 | 11.3 | 0.331 | 213 | 440 | 330 | 5.7 | 0.621 | 205 | 428 | 96 | | 2 |
| 49 | 58 | 1409 | 24.3 | 0.336 | 474 | 914 | 510 | 8.7 | 0.800 | 408 | 836 | 86 | | 3 |
| 50 | 40 | 432 | 10.8 | 0.470 | 203 | 1117 | 250 | 6.2 | 0.828 | 207 | 1043 | 101 | | 4 |
| 51 | 102 | 1315 | 12.9 | 0.385 | 506 | 1623 | 565 | 5.5 | 0.881 | 498 | 1541 | 98 | | 5 |
| 52 | 87 | 1174 | 13.5 | 0.480 | 563 | 2186 | 555 | 6.3 | 0.924 | 513 | 2054 | 91 | | 6 |
| 53 | 103 | 1215 | 11.8 | 0.431 | 524 | 2710 | 570 | 5.5 | 0.898 | 512 | 2566 | 97 | 1 + clot | 7 |
| 54 | 62 | 1339 | 21.6 | 0.402 | 538 | 3248 | 630 | 10.1 | 0.817 | 515 | 3081 | 95 | | 8 |
| 55 | 92 | 1389 | 15.1 | 0.427 | 593 | 3841 | 615 | 6.6 | 0.893 | 549 | 3630 | 92 | | 9 |
| 61 | 63 | 1197 | 19.0 | 0.302 | 361 | 4202 | 650 | 10.3 | 0.565 | 367 | 3997 | 101 | bag | 10 |
| 79 | 48 | 393 | 8.2 | 0.500 | 196 | 4398 | 215 | 4.4 | 0.830 | 176 | 4173 | 90 | | 11 |
| 80 | 29 | 365 | 12.6 | 0.421 | 153 | 4551 | 155 | 5.3 | 0.858 | 132 | 4305 | 86 | | 12 |
| 81 | 24 | 307 | 12.8 | 0.443 | 136 | 4687 | 140 | 5.8 | 0.860 | 120 | 4425 | 88 | | 13 |
| 82 | 52 | 925 | 17.8 | 0.343 | 317 | 5004 | 360 | 6.9 | 0.904 | 325 | 4750 | 102 | | 14 |
| 83 | 85 | 1096 | 12.9 | 0.474 | 519 | 5523 | 550 | 6.4 | 0.928 | 510 | 5260 | 98 | bag | 15 |
| 84 | 30 | 435 | 14.5 | 0.383 | 166 | 5689 | 190 | 6.3 | 0.790 | 150 | 5410 | 90 | bag | 16 |
| 85 | 55 | 418 | 7.6 | 0.562 | 234 | 5923 | 220 | 4.0 | 0.943 | 207 | 5617 | 88 | | 17 |
| 9 . . . 38 | 24 | 278 | 11.5 | 0.384 | 106 | 6029 | 290 | 12.0 | 0.427 | 123 | 5740 | 116 | bag | 18 |
| 9 . . . 39 | 58 | 475 | 8.1 | 0.456 | 216 | 6245 | 210 | 3.6 | 0.773 | 162 | 5902 | 75 | | 19 |
| | 58.53 | | | | | | | | | | 94.51 | 94.11 | | |
| | | | | | | | | | | | Std. Dev | 8.54 | | |

*Denotes “Validation (w/o Client)” barcode numbers

TABLE 3
Collection Volumes from CPD bags vs. Lyophilized
heparin bags for >30 cc Collections
25 Mar. 2004

| Lyophilized Bag | | CPD | |
|---|---|---|---|
| Kits | | Kits | |
| 30cc+: | | 30cc+: | |
| Mean | 69.50 | Mean | 64.96 |
| Median | 64.50 | Median | 60.50 |
| Mode | 64.00 | Mode | 55.00 |
| Sample | 476.00 | Sample | 100.00 |
| All: | | All: | |
| Mean | 65.43 | Mean | 61.20 |
| Median | 62.50 | Median | 58.00 |
| Mode | 64.00 | Mode | 55.00 |
| Sample | 520.00 | Sample | 110.00 |

TABLE 4
Pre and Post Processing Data for Lyophilized Heparin Collections

| Sample | | Pre | Pre | | Pre | Post | Post | Post | Post | | Sample |
|---|---|---|---|---|---|---|---|---|---|---|---|
| ID | Volume | WOC | NC | Pre diff | cell/mL | WOC | NC | diff. | cells/mL | % Recovery | info. |
| 4171 | 55 | 11.3 | 621 | 0.457 | 12.4 | 8.20 | 410 | 0.713 | 8.2 | 100 | |
| 4177 | 41 | 7.5 | 309 | 0.460 | 8.5 | 3.76 | 188 | 0.765 | 5.2 | 100 | |
| 4178 | 174 | 10.0 | 1736 | 0.529 | 10.9 | 20.80 | 1040 | 0.713 | 6.5 | 80 | |
| 4179 | 115 | 16.8 | 1932 | 0.342 | 18.4 | 20.90 | 1045 | 0.580 | 9.9 | 91 | |
| 4180 | 74 | 9.9 | 735 | 0.369 | 11.4 | 8.85 | 442 | 0.566 | 6.9 | 92 | |
| 4218 | 22 | 8.5 | 187 | 0.460 | 11.0 | 2.08 | 104 | 0.734 | 6.1 | 88 | |
| 4219 | 74 | 15.9 | 1176 | 0.469 | 18.3 | 13.60 | 680 | 0.832 | 10.6 | 100 | |
| 4220 | 32 | 11.6 | 371 | 0.548 | 13.7 | 4.64 | 232 | 0.793 | 8.6 | 90 | |
| 4221 | 37 | 12.2 | 451 | 0.381 | 14.0 | 4.50 | 225 | 0.822 | 6.0 | 100 | |
| 4222 | 50 | 18.9 | 945 | 0.416 | 21.0 | 10.20 | 510 | 0.738 | 11.3 | 95 | |
| 4223 | 44 | 9.1 | 402 | 0.482 | 10.3 | 5.04 | 252 | 0.745 | 6.4 | 96 | |
| 4224 | 53 | 17.6 | 932 | 0.386 | 20.2 | 8.49 | 424 | 0.815 | 9.2 | 96 | |
| 4236 | 22 | 10.4 | 228 | 0.547 | 13.4 | 3.81 | 190 | 0.659 | 11.1 | 100 | |
| 4237 | 28 | 10.7 | 299 | 0.451 | 13.0 | 4.60 | 230 | 0.579 | 10.0 | 99 | |
| 4240 | 68 | 11.6 | 765 | 0.326 | 13.6 | 5.91 | 295 | 0.841 | 5.2 | 99 | |
| 4277 | 20 | 9.9 | 196 | 0.418 | 13.2 | 3.10 | 155 | 0.605 | 10.3 | 100 | |
| 4288 | 62 | 21.5 | 1333 | 0.304 | 23.3 | 10.50 | 525 | 0.699 | 9.2 | 90 | 1 + clots |

TABLE 5
(including chart)
Pre and Post Processing Data for Liquid Heparin in Syringe and Volume Collection Histogram
5 ml Heparin Per Syringe Storage

| | | | | | | |
|---|---|---|---|---|---|---|
| Vol w/o Hep | Pre NC Count | Pre % MNC | Post NC Count | Post % MNC | Percent Recovered | Cells per ml |

All Volumes

| | | | | | | | |
|---|---|---|---|---|---|---|---|
| Mean | 71.06 | 1002.97 | 41.33% | 492.71 | 78.35% | 94.34 | 6.92 |
| Median | 68 | 917 | 40.40% | 445 | 82.20% | 94.485 | 6.47 |
| Mode | 60 | 756 | 40% | 380 | 80% | 96.25 | 5 |
| Size | 9902 | 9902 | 9902 | 9902 | 9902 | 9902 | 9902 |

Greater than or Equal to 30 cc

| | | | | | | | |
|---|---|---|---|---|---|---|---|
| Mean | 73.80 | 1042.93 | 41.21% | 512.38 | 78.73% | 94.98 | 6.95 |
| Median | 70 | 949 | 40.30% | 460 | 82.40% | 94.78 | 6.52 |
| Mode | 60 | 756 | 40% | 380 | 80% | 96.25 | 5 |
| Size | 9387 | 9387 | 9387 | 9387 | 9387 | 9387 | 9387 |

Failure Rate 5.20%

TABLE 6
(including chart):
Pre and Post Processing Data for Lyophilized Heparin in Syringe and Volume Collection Histogram
Lyophilized Syringe Storage

| | | | | | | |
|---|---|---|---|---|---|---|
| Vol w/o Hep | Pre NC Count | Pre % MNC | Post NC Count | Post % MNC | Percent Recovered | Cells per ml |

All Volumes

| | | | | | | | |
|---|---|---|---|---|---|---|---|
| Mean | 67.45 | 857.58 | 42.50% | 426.45 | 79.63% | 94.66 | 6.33 |
| Median | 64 | 774.7 | 41.07% | 383.75 | 81.50% | 95.03 | 5.98 |
| Mode | 60 | 780 | 40% | 505 | 80% | 95.18 | 5 |
| Size | 9926 | 9926 | 9926 | 9926 | 9926 | 9926 | 9926 |

Greater than or Equal to 30 cc

| | | | | | | | |
|---|---|---|---|---|---|---|---|
| Mean | 71.04 | 904.00 | 42.48% | 449.11 | 79.91% | 94.70 | 6.33 |
| Median | 66 | 810.58 | 41.06% | 400.75 | 81.70% | 95.05 | 5.98 |
| Mode | 60 | 780 | 40% | 505 | 80% | 95.18 | 5 |
| Size | 9224 | 9224 | 9224 | 9224 | 9224 | 9224 | 9224 |

Failure Rate 7.07%

TABLE 7
(including chart): Pre and Post Processing Data for
Liquid Heparin in Bag and Volume Collection Histogram
30 ml Heparin Bag Storage

| | | | | | | |
|---|---|---|---|---|---|---|
| Vol w/o Hep | Pre NC Count | Pre % MNC | Post NC Count | Post % MNC | Percent Recovered | Cells per ml |

All Volumes

| | | | | | | | |
|---|---|---|---|---|---|---|---|
| Mean | 62.35 | 934.46 | 45.34% | 347.15 | 77.04% | 85.89 | 5.41 |
| Median | 59 | 854.875 | 43.10% | 300 | 81.40% | 69.815 | 5.08 |
| Mode | 50 | 1080 | 40% | 150 | 80% | 51.5 | 5 |
| Size | 766 | 766 | 766 | 766 | 765 | 766 | 766 |

Greater than or Equal to 30 cc

| | | | | | | | |
|---|---|---|---|---|---|---|---|
| Mean | 67.93 | 1021.74 | 44.19% | 380.80 | 79.42% | 71.08 | 5.49 |
| Median | 63 | 915 | 42.75% | 333.75 | 82.40% | 70.715 | 5.255 |
| Mode | 50 | 1080 | 40% | 200 | 80% | 51.5 | 5 |
| Size | 678 | 678 | 678 | 678 | 678 | 678 | 678 |
| Failure Rate | 11.49% | | | | | | |

TABLE 8
(including chart):
Pre and Post Processing Data for Lyophilized Heparin in Bag and Volume Collection Histogram
Lyophilized Bag Storage

| | | | | | | |
|---|---|---|---|---|---|---|
| Vol w/o Hep | Pre NC Count | Pre % MNC | Post NC Count | Post % MNC | Percent Recovered | Cells per ml |

All Volumes

| | | | | | | | |
|---|---|---|---|---|---|---|---|
| Mean | 62.96 | 794.35 | 42.45% | 395.36 | 80.11% | 97.84 | 6.36 |
| Median | 60 | 705.76 | 41.54% | 357 | 82.00% | 97.53 | 5.97 |
| Mode | 47 | 660.8 | 40.00% | 530 | 80.00% | 98.26 | 7.5 |
| Size | 1783 | 1783 | 1783 | 1783 | 1783 | 1783 | 1783 |

Greater than or Equal to 30 cc

| | | | | | | | |
|---|---|---|---|---|---|---|---|
| Mean | 67.80 | 858.40 | 42.21% | 425.06 | 80.76% | 97.59 | 6.34 |
| Median | 63 | 763.83 | 41.40% | 380.5 | 82.60% | 97.37 | 5.93 |
| Mode | 47 | 660.8 | 40.00% | 530 | 80.00% | 98.26 | 5 |
| Size | 1599 | 1599 | 1599 | 1599 | 1599 | 1599 | 1599 |

Failure Rate 10.32%

TABLE 9
Pre and Post Processing Data for Liquid Heparin
in Bag and Volume Collection Histogram
CPD Bag Storage

| | | | | | | |
|---|---|---|---|---|---|---|
| Vol w/o Hep | Pre NC Count | Pre % MNC | Post NC Count | Post % MNC | Percent Recovered | Cells per ml |

All Volumes

| | | | | | | | |
|---|---|---|---|---|---|---|---|
| Mean | 60.32 | 894.79 | 46.38% | 422.65 | 73.46% | 72.781 | 6.75 |
| Median | 57 | 803 | 45.50% | 379 | 74.90% | 74.06 | 6.42 |
| Mode | 55 | 552 | 43.00% | 345 | 82.90% | 86.63 | 6.25 |
| Size | 139 | 139 | 139 | 139 | 139 | 139 | 139 |

Greater than or Equal to 30 cc

| | | | | | | | |
|---|---|---|---|---|---|---|---|
| Mean | 64.14 | 952.88 | 46.44% | 450.88 | 74.16% | 74.33 | 6.83 |
| Median | 60.5 | 820 | 46.15% | 392.5 | 75.60% | 74.94 | 6.48 |
| Mode | 55 | 552 | 43.00% | 170 | 82.90% | 59.5 | 6.25 |
| Size | 126 | 126 | 126 | 126 | 126 | 126 | 126 |
| Failure Rate | 9.35% | | | | | | |

TABLE 10
Comparison of Low Volume Collections in Syringes with Liquid Heparin and Lyophilized Heparin
From May 16, 2003 through Jul. 19, 2004

| | | | | | | |
|---|---|---|---|---|---|---|
| Quantity of | Lab: | Lab: | | Lab: | Lab: | Lab: |
| Blood Received | TNCPre | MNC % Pre | Number of TNC | MNC % Post | cells_per_ml | cnRecovery % MNC |

Liquid Heparin
Sample Size: 583 (collections less than or equal to 30 ccs/heparin at least about 25% of total collection volume)

| | | | | | | | |
|---|---|---|---|---|---|---|---|
| Mean | 29.33 | 394.02 | 41.57% | 197.90 | 69.65% | 6.64 | 85.54 |
| Median | 30.00 | | | | | | |
| Mode | 45.00 | | | | | | |

Lyophilized Heparin
Sample Size: 1469 (collections less than or equal to 30 ccs*)

| | | | | | | | |
|---|---|---|---|---|---|---|---|
| Mean | 36.69 | 466.28 | 42.34% | 237.63 | 78.08% | 6.45 | 96.14 |
| Median | 37.00 | | | | | | |
| Mode | 39.00 | | | | | | |

*As lyophilized collections will not have a measurement for % heparin in each sample, the following parameters were used to generate a comparable data set: Using the heparin group as a benchmark, collections where heparin comprised of 25% or more of the total volume, for the number of syringe used, the volume ranged from:
1 Syringe Collections: Coll Vol 0-20
2 Syringe Collections: Coll Vol 0-40
3 Syringe Collections: Coll Vol 0-60

Thus, lyophilized collections whereby the collection volume, based on # of syringes used, fell in the above ranges, the data was used for comparison.

1: A kit for collecting a desired amount cord blood comprising:
at least one collecting receptacle for collecting cord blood; and
a pre-measured non-liquid anticoagulant;
wherein the anticoagulant is pre-loaded into the receptacle.

2: The kit of claim 1 wherein the collecting receptacle is selected from the group consisting of a syringe, bag, beaker and pouch.

3: The kit of claim 1 wherein the anticoagulant is selected from the group consisting of heparin, heparin type additives, citrates, EDTA (ethylenediaminetetraacetic acid), and oxalates.

4: The kit of claim 1 wherein the anticoagulant is lyophilized or in powder form.

5: The kit of claim 1 wherein the anticoagulant is lyophilized heparin or sodium citrate.

6: The kit of claim 1 wherein the collecting receptacle is a bag and the non-liquid anticoagulant is lyophilized heparin,
wherein the lyophilized heparin is in the amount of 5 or about 5 heparin pellets.

7: The kit of claim 1 wherein the collecting receptacle is a syringe and the non-liquid anticoagulant is lyophilized heparin,
wherein the lyophilized heparin is in the amount of 5 or about 5 heparin pellets.

8: The kit of claim 1 wherein the collecting receptacle is a syringe, the non-liquid anticoagulant is lyophilized heparin,
wherein the lyophilized heparin is in the amount of 5 or about 5 heparin pellets, and the amount of cord blood collected is less than or equal to 30 ccs.

9: The kit of claim 1 further comprising at least one of the following components:
one or more containers for maternal blood collection;
one or more sterile wipes;
one or more needles;
one or more trays;
one or more identification labels;
one or more instructions;
one or more gauze pads;
one or more needle holders; and
wherein all of the components of the kit are stored in a sterile container prior to use.

10: The kit of claim 1 wherein the anti-coagulant is pre-loaded in a sterile manufacturing environment.

11: A kit for collecting a desired amount of cord blood comprising:
at least one collecting receptacle for collecting cord blood; and
a pre-measured lyophilized anticoagulant;
wherein the anticoagulant is pre-loaded into the receptacle, and wherein the anticoagulant is heparin or sodium citrate; and
the collecting receptacle is a syringe or a bag; and
wherein the amount of cord blood collected is less than or equal to 30 ccs.

12: The kit of claim 11 wherein the pre-measured lyophilized anticoagulant is 5 or about 5 heparin pellets.

13: The kit of claim 11 further comprising at least one of the following components:
one or more containers for maternal blood collection;
one or more sterile wipes;
one or more needles;
one or more trays;
one or more identification labels;
one or more instructions;
one or more gauze pads;
one or more needle holders; and
wherein all of the components of the kit are stored in a sterile container prior to use.

14: The kit of claim 11 wherein the anti-coagulant is pre-loaded in a sterile manufacturing environment.

15: A method for collecting cord blood comprising: collecting a desired amount of cord blood from the umbilical cord using a collection receptacle of the invention, wherein the collection receptacle is pre-loaded with a pre-measured amount of non-liquid anticoagulant.

16: The method of claim 15 wherein the collecting receptacle is selected from the group consisting of a syringe, bag, beaker and pouch.

17: The method of claim 15 wherein the anticoagulant is selected from the group consisting of heparin, heparin type additives, citrates, EDTA (ethylenediaminetetraacetic acid), and oxalates.

18: The method of claim 15 wherein the anticoagulant is lyophilized heparin or sodium citrate.

19: The method of claim 18 wherein the desired amount of collected blood is less than or equal to about 30 ccs and the % MNC recovery is at least about 95%.

20: The method of claim 15 wherein the anticoagulant is lyophilized heparin or sodium citrate and the collecting receptacle is a syringe or a bag; and wherein the amount of cord blood collected is less than or equal to 30 ccs.

21: The method of claim 15 wherein the anti-coagulant is pre-loaded in a sterile manufacturing environment.
